(19) United States
(12) Patent Application Publication
(10) Pub. No.: US 2003/0181825 A1
(43) Pub. Date: Sep. 25, 2003

(54) Blood sample syringe

(76) Inventor: Klaus Büttner, (US)

Correspondence Address: VENABLE, BAETJER, HOWARD AND CIVILETTI, LLP, P.O. BOX 34385, WASHINGTON, DC, 20043-9998, US

(21) Appl. No.: 10/362,920

(22) PCT Filed: Jun. 12, 2002
(86) PCT No.: PCT/EP02/06413

(30) Foreign Application Priority Data
Jun. 30, 2001 (DE) 201-10-872.0

(51) Int. Cl. A61B 5/00; B65D 81/00

(52) U.S. Cl. 600/576

(57) ABSTRACT

The invention relates to a blood collection syringe which has a cylindrical blood reception space accessible by way of a hollow needle with a piston component movable inside such syringe with a plunger component extending to the exterior. The plunger component is configured so as to be adjacent to the piston component and to have a break-off point for the purpose of dividing the plunger component into a removable component and a component rigidly connected to the piston component. The piston is provided with a membrane or a perforation point for a serum collection syringe. At the break-off point is an access channel for the serum collection needle, this channel extending to the perforation point.

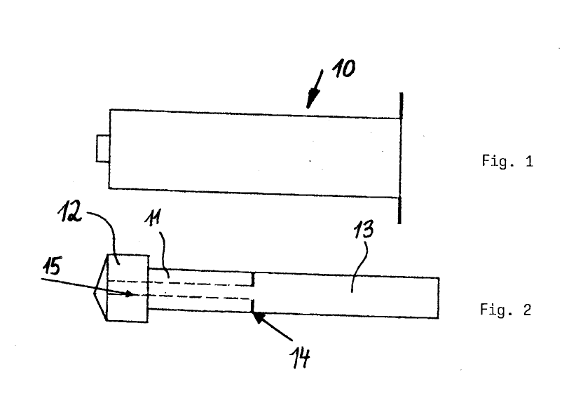

The invention relates to a blood collection syringe having a cylindrical blood reception space accessible by a hollow needle with a piston component movable inside such space by a plunger component extending to the exterior.

For autologous transfusion or blood transfusion of an individual's own blood or prepared components of the patient's own blood, the blood obtained from a patient in advance of a scheduled surgical intervention is centrifuged and withdrawn in syringes, so that the individual's own blood may be introduced into him at shorter or longer intervals after the surgical intervention, above all in order to compensate for copious blood losses. The advantages of this process are to be viewed as the circumstance that no transfusion incidents determined by immunity can occur, or that no transfer of alien pathogens is possible. However, special purity conditions must be strictly adhered to from blood collection to retransfusion of the blood serum.

In known processes the blood collection syringe is employed to draw blood from a patient and the syringe is transported to a laboratory in a refrigerated container. In the laboratory the blood is removed from the collection syringe and transferred to centrifuge tubes. The centrifuge tubes are placed in a centrifuge and serum may be removed from the upper area of the centrifuge tubes after centrifuging by means of a serum collection needle, corpuscular components remaining in the lower area of the centrifuge tubes. The centrifuge tubes must be opened during withdrawal of the serum, so that in the aggregate two conflicting processes subject to the most rigorous clinical demands are carried out, namely filling of the centrifuge tubes with blood and removal of the serum.

The present invention is introduced at this point, with the aim of configuring a blood collection syringe so that the conditions of a closed system are present over the entire process from withdrawal of blood to transfer of the serum to suitable serum syringes.

This is achieved in particular in that, in the case of a blood collection syringe of the kind referred to above, the plunger is designed with a break-off point in order to divide the plunger into a removable part and a part rigidly connected to the piston, the piston component being configured with a transparent area for the serum collection needle, so that the blood withdrawn during centrifuging up to the time of withdrawal of the blood serum need not be removed from the syringe.

In use of the blood collection syringe claimed for the invention the blood withdrawn remains in the blood collection syringe, specifically until the separated serum may be removed by a serum collection needle. Transfer processes required in use of the known blood collection syringes may be dispensed with.

In order for it not to be necessary to configure the centrifuges with excessively great dimensions, in the blood collection syringe claimed for the invention the plunger component is divided into two parts or may be divided into two parts, so that after withdrawal of blood, that is, retraction of the plunger component, the longitudinal extension is reduced. The radial extension of the blood collection syringe in the centrifuge is also reduced; that is to say, centrifuges in use up to the present may continue to be used in conjunction with the blood collection syringe claimed for the invention.

After the centrifuging process has been completed the serum collection needle may be inserted into the interior of the blood collection syringe, appropriate walls in the piston component or a membrane provided there being perforated. Access for the serum collection needle is cleared in that the two plunger component parts are separated from each other at the designed break-off point.

In the aggregate the blood collection syringe claimed for the invention forms a closed system; up to the present this has not been the case.

The two parts of the plunger component may be injection molded with plastic with the piston component to form one piece. It is also conceivable that the two plunger component parts could be joined by some sort of breakable connection before the blood collection syringe is inserted into the centrifuge.

Other advantageous embodiments are specified in the dependent claims.

The sole FIGURE of the drawing illustrates a collection syringe as claimed for the invention, that is, the cylinder proper plus the plunger component.

In the FIGURE the cylinder of a blood collection syringe is identified as 10. In this cylinder the piston component 12 may be moved back and forth, this movement being effected by the extruded plunger component 11, 13. The two plunger component parts 11 and 13 may be separated at a break-off point 14, the break-off point being oriented nearer the longitudinal extension of the piston component in the embodiment illustrated. The optimal location is oriented rather toward the vicinity of the piston component, since when the interior of the cylinder is filled the blood collection syringe assumes its shortest extension after separation of part 13 from part 14.

The number 15 identifies a channel which is open in the area of the break-off point 14, is situated centrally in the plunger component 11, and ends in the frontal area in the front end of the piston component 12. Either a thin wall of the piston component or a rubber-like membrane is situated there, so that this location may be perforated at this point as a result of introduction of the serum collection syringe and so that the blood inside the blood collection syringe or the serum present there may be withdrawn.

During use the plunger component 11, 13 is inserted fully into the cylinder 10 from right to left as seen along the line of sight in the FIGURE. As a result of movement of the piston component 12 to the right blood may now be drawn into the interior of the cylinder 10. The plunger component 13 accordingly moves an increasing distance from the interior of the cylinder 10. The break-off point 14 may now be used to separate the plunger component 13 from the plunger component 11. While in this state the blood collection syringe is introduced into the centrifuge and the serum, after being separated by the centrifugal forces, may be removed from the interior of the cylinder 10 by introduction of a serum collection syringe through the channel 15 from the right. An opening is produced in the area of the break-off point as a result of separation of plunger components 11 and 13 from each other. At the front end of the piston component 12 the tip of the serum syringe perforates the membrane present there and the serum may now be withdrawn without difficulty.

It will be seen that a closed system is present from blood collection to serum removal.

1. A blood collection syringe having a blood receiving space accessible by way of a hollow needle, with a piston component movable in such space, the piston component having a plunger component extending to the exterior, characterized in that the plunger component (11, 13) is adjacent to the piston component (12) and is configured with a break-off point (14) or similar detachable connection of two plunger components (11, 13) in order to separate the plunger component into a removable part (13) and with a part (11) rigidly connected to the piston component (12), in that the piston component (12) is provided with a membrane or a perforation point for a serum collection needle, and in that the break-off point (14) is in the form of an access channel (15) for the serum collection needle, which channel extends to the perforation point.

2. The blood collection syringe as claimed in claim 1, wherein the piston component (12) and the plunger component (11, 13) are injection-molded as one piece of plastic.

3. The blood collection syringe as claimed in one of claims 1 or 2, wherein the perforation point and the access channel (15) are configured to be central relative to the longitudinal extension of the syringe.

4. The blood collection syringe as claimed in one of claims 1 to 3, wherein the serum collection needle is mounted on a separator piece from which the collected serum is introduced into a plurality of syringes.
